(19) United States
(12) Patent Application Publication
(10) Pub. No.: US 2026/0183069 A1
(43) Pub. Date: Jul. 2, 2026

(54) TOOL CHANGING SYSTEM FOR SURGICAL ROBOT OPERATION

(71) Applicant: FoxEyes Corporation, Plano, TX (US)

(72) Inventors: HONGWON YOON, Allen, TX (US); Seulki KIM, Uiwang-si (KR); Ting-Yun FANG, Taipei City (TW); Jieun SANG, Irvine, CA (US)

(73) Assignee: FoxEyes Corporation, Plano, TX (US)

(21) Appl. No.: 19/302,997

(22) Filed: Aug. 18, 2025

Related U.S. Application Data
(60) Provisional application No. 63/739,303, filed on Dec. 27, 2024.

(51) Int. Cl. A61B 34/30; A61B 17/00; A61B 34/20; A61B 34/37; A61B 90/00; B25J 15/04

(52) U.S. Cl. CPC A61B 34/30; A61B 34/20; A61B 34/37; A61B 90/37; B25J 15/04; A61B 2017/00477

(57) ABSTRACT

A surgical robot apparatus includes a movable body portion, a robot arm disposed on the body portion to perform a predetermined operation, a first tool changer coupled to the robot arm, a second tool changer to which a medical tool is coupled and which is detachable from the first tool changer, a hanger on which the second tool changer is mounted, and a control unit controlling an operation of the robot arm.

### CROSS-REFERENCE TO RELATED PATENT APPLICATION

This application claims the benefit of U.S. Provisional Application No. 63/739,303, filed on Dec. 27, 2024, the disclosure of which is incorporated herein in its entirety by reference.

### TECHNICAL FIELD

The present disclosure relates to a tool changing system for surgical robot operation.

### BACKGROUND

Surgical operations rely on the manual skills of a surgeon, and there is a problem in that the precision of surgery decreases due to hand tremors or fatigue of a human. In addition, in the case of open surgery, there is a problem in that the recovery time is long and the risk of infection increases.

In order to solve such problems, various surgical robots are being developed. Surgical robots may perform precise and repetitive surgical operations. In particular, surgical robots may enable minimally invasive surgery (MIS), thereby shortening the patient's recovery time, reducing complications, and improving surgical precision.

As an example, a surgical robot may include a surgical medical tool coupled to an end of a robot arm, and a person may operate the robot arm to perform surgery. However, there is inconvenience in that only one surgical medical tool is coupled to the robot arm, requiring frequent replacement of the medical tool. In addition, there is a problem in that contamination may occur during the process of replacing the medical tool.

The above information disclosed in the technology forming the background of the present invention is only for improving understanding of the background of the invention, and therefore may include information that does not constitute prior art.

### SUMMARY

The present disclosure provides a surgical robot apparatus for solving the above problems.

However, the technical problem to be solved by the present invention is not limited to the above-described problems, and other problems not mentioned will be clearly understood by those skilled in the art from the description of the invention set forth below.

According to one embodiment of the present invention to solve the above technical problem, a surgical robot apparatus may include a movable body portion, a robot arm disposed on the body portion to perform a predetermined operation, a first tool changer coupled to the robot arm, a second tool changer to which a medical tool is coupled and which is detachable from the first tool changer, a hanger on which the second tool changer is mounted, and a control unit for controlling the operation of the robot arm.

In some embodiments, the control unit may control the operation of the robot arm to couple the first tool changer to the second tool changer according to predetermined position data of the hanger.

In some embodiments, in a state where the first tool changer and the second tool changer are coupled, the control unit may control the operation of the robot arm according to the predetermined position data of the hanger to mount the second tool changer on the hanger and then separate the first tool changer from the second tool changer.

In some embodiments, the first tool changer may include a first tool changer body coupled to the robot arm, and a coupling protrusion protruding from the first tool changer body, inserted into the second tool changer, and including a coupling groove coupled to the second tool changer.

In some embodiments, the second tool changer may include a second tool changer body to which the medical tool is coupled and which includes an insertion hole into which the coupling protrusion is inserted, and a locking part provided on the second tool changer body, coupled to the hanger when the hanger is disposed, and coupled to the first tool changer when detached from the hanger.

In some embodiments, the locking part may include a locking lever rotatably disposed on the second tool changer body that is rotationally constrained by a locking protrusion provided on the hanger, a locking block arranged to be slidingly movable on the second tool changer body and inserted into or separated from the coupling groove in response to the rotation of the locking lever, and an elastic member engaged with the locking block and exerting elastic force to enable insertion of the locking block into the coupling groove.

In some embodiments, the surgical robot apparatus may further include a coupling block to which the second tool changer is coupled at one side and to which at least one medical tool is coupled at the other side.

In some embodiments, the surgical robot apparatus may further include a support portion provided on the body portion and to which the robot arm is coupled, and at least one slide bar coupled to the support portion to slide in parallel to the direction in which the body portion moves, wherein the hanger is detachably coupled to the slide bar.

In some embodiments, the slide bar may include a plurality of through-holes formed along a length direction, and the hanger may be detachably coupled to the slide bar by being coupled to or separated from a coupling member inserted into the plurality of through-holes.

In some embodiments, the surgical robot apparatus may further include a support portion provided on the body portion and to which the robot arm is coupled, a coupling plate coupled to the support portion, at least one slide portion detachably coupled to the coupling plate and sliding, and a hanger coupling portion detachably coupled to the slide portion and provided with the hanger.

In some embodiments, the slide portion may include a slide body including a coupling protrusion coupled to a coupling hole formed in the coupling plate, a slide bar including a guide rail coupled to the slide body and sliding, and a coupling hook provided on the slide bar and coupled to a hook coupling groove formed at both ends of the hanger coupling portion.

In some embodiments, the coupling protrusion may include a first coupling protrusion formed at one side of the slide body and a second coupling protrusion formed at the other side of the slide body, the coupling hole may include a first coupling hole formed to penetrate from one side of the coupling plate to the outside and a second coupling hole formed to penetrate from the other side of the coupling plate in a direction perpendicular to the first coupling hole, and after the first coupling protrusion is inserted into the first coupling hole, the slide body may rotate to insert the second coupling protrusion into the second coupling hole.

In some embodiments, the surgical robot apparatus may further include a coupling member coupled to the coupling plate and inserted into a pin coupling groove formed in the slide body, and when the second coupling protrusion is inserted into the second coupling hole, the coupling pin may be inserted into the pin coupling groove to restrain the slide body from rotating.

In some embodiments, the surgical robot apparatus may further include a vision marker provided on at least one of the hanger and the second tool changer and indicating the type of the medical tool coupled to the second tool changer, and a vision sensor recognizing the vision marker.

In some embodiments, the control unit may control the operation of the robot arm to couple the first tool changer to the second tool changer by identifying the position of the hanger from the vision marker photographed by the vision sensor.

In some embodiments, the hanger may be disposed in plurality, a plurality of second tool changers each coupled with different medical tools may be coupled to the plurality of hangers, and a plurality of vision markers may be provided on the plurality of hangers.

In some embodiments, the control unit may identify the positions and alignment directions of the plurality of hangers from the plurality of vision markers photographed by the vision sensor.

In some embodiments, the control unit may identify the types of the plurality of medical tools disposed on the plurality of hangers from the plurality of vision markers provided on the plurality of hangers.

In some embodiments, the control unit may control the operation of the robot arm to couple the first tool changer to the second tool changer coupled with the medical tool necessary for a predetermined operation among the plurality of second tool changers.

In some embodiments, the control unit may control the operation of the robot arm such that, when the use of the medical tool of the second tool changer coupled to the first tool changer is completed, the second tool changer is coupled to the hanger, and the first tool changer is coupled to a second tool changer to which the medical tool necessary for the set next operation is coupled.

According to an embodiment of the present invention, the surgical robot apparatus is equipped with a tool changer on the robot arm and the medical tool, enabling the automatic replacement of medical tools through the operation of the robot arm, thereby improving work convenience.

According to an embodiment of the present invention, the surgical robot apparatus may replace medical tools by the operation of the robot arm without direct contact by the operator, thereby effectively preventing the risk of infection.

According to an embodiment of the present invention, the surgical robot apparatus may configure the hanger on which the medical tool is mounted to be detachable, enabling the hanger to be separately sterilized, thereby effectively preventing the risk of infection.

According to an embodiment of the present invention, the surgical robot apparatus may configure the hanger on which the medical tool is mounted to be movable in a sliding manner, thereby easily adjusting the position of the hanger and improving usability.

According to an embodiment of the present invention, the surgical robot apparatus may identify the positions of the hanger and/or the tool changer and the type of the medical tool by recognizing the vision marker provided on the hanger and/or the tool changer with a vision sensor provided on the robot arm.

According to an embodiment of the present invention, the surgical robot apparatus may automatically replace the medical tool required for surgery by identifying the type of the medical tool coupled to the tool changer through the vision marker and the vision sensor.

The effects of the present disclosure are not limited to the effects mentioned above. Other effects not mentioned will be clearly understood by one of ordinary skill in the art from the statements of the claims.

### BRIEF DESCRIPTION OF THE DRAWINGS

Embodiments will be described below with reference to the attached drawings, in which like reference numerals denote like elements but are not limited thereto.

FIG. 1 is a perspective view illustrating an example of a surgical robot apparatus according to an embodiment of the present disclosure.

FIG. 2 is a perspective view illustrating an enlarged view of a robot arm and a hanger in the surgical robot apparatus according to an embodiment of the present disclosure.

FIG. 3 is a perspective view illustrating an example in which the robot arm and the medical tool are coupled through a tool changer in the surgical robot apparatus according to an embodiment of the present disclosure.

FIG. 4 is a perspective view illustrating an example in which the medical tool coupled to the robot arm is separated from the hanger in the surgical robot apparatus according to an embodiment of the present disclosure.

FIG. 5 is a perspective view illustrating another example of the hanger in the surgical robot apparatus according to an embodiment of the present disclosure.

FIG. 6 is an enlarged view illustrating an example of the first tool changer coupled to the robot arm and the second tool changer coupled to the medical tool in the surgical robot apparatus according to an embodiment of the present disclosure.

FIG. 7 is a cross-sectional view illustrating an example in which the first tool changer is disposed on the second tool changer coupled to the hanger in the surgical robot apparatus according to an embodiment of the present disclosure.

FIG. 8 is a cross-sectional view illustrating an example in which the second tool changer is separated from the hanger while being coupled to the first tool changer in the surgical robot apparatus according to an embodiment of the present disclosure.

FIG. 9 is a perspective view illustrating an example of a coupling block integrally coupled with a medical tool in the surgical robot apparatus according to an embodiment of the present disclosure.

FIG. 10 is a perspective view illustrating an example of a coupling block to which the medical tool is detachably coupled in the surgical robot apparatus according to an embodiment of the present disclosure.

FIG. 11 is a perspective view illustrating an example in which the medical tool is coupled to the coupling block of FIG. 10.

FIG. 12 is a perspective view illustrating an example of a coupling block integrally coupled with two medical tools in the surgical robot apparatus according to an embodiment of the present disclosure.

FIG. 13 is a perspective view illustrating an example in which a plate rotates to change the position of the hanger in the surgical robot apparatus according to an embodiment of the present disclosure.

FIG. 14 is a side view illustrating an example in which an actuator and a lifter are provided in the body portion of the surgical robot apparatus according to an embodiment of the present disclosure.

FIG. 15 is a side view illustrating an example in which the actuator and the lifter are operated in the surgical robot apparatus according to an embodiment of the present disclosure.

FIG. 16 is a perspective view illustrating an example in which a vision sensor and a vision marker are provided in the surgical robot apparatus according to an embodiment of the present disclosure.

FIG. 17 is a perspective view illustrating an example in which a vision sensor is provided on the robot arm in the surgical robot apparatus according to an embodiment of the present disclosure.

FIG. 18 is a perspective view illustrating an example in which a vision marker is provided on the hanger in the surgical robot apparatus according to an embodiment of the present disclosure.

FIG. 19 is a perspective view illustrating an example in which the vision sensor recognizes the vision marker to identify the position of the hanger in the surgical robot apparatus according to an embodiment of the present disclosure.

FIG. 20 is a perspective view illustrating an example in which the vision sensor recognizes the vision marker to identify the type of the medical tool provided on the hanger in the surgical robot apparatus according to an embodiment of the present disclosure.

FIG. 21 is a perspective view illustrating an example in which the first tool changer is coupled to the second tool changer after recognizing the vision marker in the surgical robot apparatus according to an embodiment of the present disclosure.

FIG. 22 is a perspective view illustrating an example in which the medical tool coupled to the robot arm is separated from the hanger in the surgical robot apparatus according to an embodiment of the present disclosure.

FIG. 23 is a perspective view illustrating an example in which a vision marker is provided on the second tool changer in the surgical robot apparatus according to an embodiment of the present disclosure.

FIG. 24 is a perspective view illustrating an example in which vision markers are provided on the hanger and the second tool changer in the surgical robot apparatus according to an embodiment of the present disclosure.

FIG. 25 is a perspective view illustrating an example of a slide bar to which the hanger is detachably coupled in the surgical robot apparatus according to an embodiment of the present disclosure.

FIG. 26 is a perspective view illustrating an example in which the slide bar has been slid in the surgical robot apparatus according to an embodiment of the present disclosure.

FIG. 27 is a perspective view illustrating an example in which the hanger is coupled to the slidably moved slide bar in the surgical robot apparatus according to an embodiment of the present disclosure.

FIG. 28 is a perspective view illustrating an example in which the hanger is coupled to the slide bar in the surgical robot apparatus according to an embodiment of the present disclosure.

FIG. 29 is a perspective view illustrating an example of a slide portion to which the hanger is detachably coupled in the surgical robot apparatus according to an embodiment of the present disclosure.

FIG. 30 is a plan view illustrating an example of a coupling plate to which the slide portion is detachably coupled in the surgical robot apparatus according to an embodiment of the present disclosure.

FIG. 31 is an exploded cross-sectional view illustrating an example of the coupling plate, the slide portion, the hanger coupling portion, and the hanger in the surgical robot apparatus according to an embodiment of the present disclosure.

FIG. 32 is a perspective view illustrating an example in which the slide portion is coupled to the coupling plate in the surgical robot apparatus according to an embodiment of the present disclosure.

FIG. 33 is a perspective view illustrating an example in which the slide portion is coupled to the coupling plate in the surgical robot apparatus according to an embodiment of the present disclosure.

FIG. 34 is a side view illustrating an example in which the slide bar is moved in the slide portion coupled to the coupling plate in the surgical robot apparatus according to an embodiment of the present disclosure.

### DETAILED DESCRIPTION

Hereinafter, preferred embodiments of the present invention will be described in detail with reference to the accompanying drawings. Prior to this, terms or words used in the present specification and claims should not be construed as being limited to the usual or dictionary meaning, but should be interpreted as meanings and concepts consistent with the technical idea of the present invention based on the principle that the inventor can appropriately define the concept of the term to describe his own invention in the best way. Therefore, the embodiments described in the present specification and the configurations illustrated in the drawings are only the most preferred embodiments of the present invention and do not represent all of the technical ideas of the present invention, so it should be understood that various equivalents and modifications capable of replacing these at the time of filing the application may exist.

In addition, when used in the present specification, “comprise” and/or “including” means specifying the presence of the stated shapes, numbers, steps, operations, members, elements, and/or groups thereof, but does not exclude the presence or addition of one or more other shapes, numbers, operations, members, elements, and/or groups thereof.

In addition, in order to aid understanding of the invention, the accompanying drawings may not be depicted to scale, and the dimensions of some components may be exaggerated. Further, the same reference numerals may be assigned to the same components in different embodiments.

The statement that two comparative objects are “identical” means that they are “substantially identical.” Thus, substantial identity may include cases having a deviation considered low in the art, for example, within 5% deviation. In addition, the statement that a certain parameter is uniform in a certain region may mean uniform in an average view.

Although first, second, etc. are used to describe various components, these components are not limited by these terms. These terms are only used to distinguish one component from another component, and unless specifically stated otherwise, the first component may also be the second component.

Throughout the specification, unless specifically stated otherwise, each component may be singular or plural.

The statement that any configuration is disposed “on” or “under” a component may mean that any configuration is disposed in contact with the upper or lower surface of the component, but may also mean that another configuration is interposed between the component and the configuration disposed on (or under) the component.

In addition, when a component is described as being “connected,” “coupled,” or “attached” to another component, it should be understood that the components may be directly connected or coupled to each other, but another component may be interposed therebetween, or each component may be connected, coupled, or attached to another component through another component. In addition, when a part is electrically connected to another part, it includes not only cases where it is directly connected but also cases where another element is interposed in between.

Throughout the specification, when “A and/or B” is used, unless specifically stated otherwise, it means A, B, or A and B. That is, “and/or” includes all combinations or any combination of the enumerated multiple items. When “C to D” is used, unless specifically stated otherwise, it means greater than or equal to C and less than or equal to D.

The terms used in the present specification are for describing embodiments of the present disclosure and are not intended to limit the present disclosure.

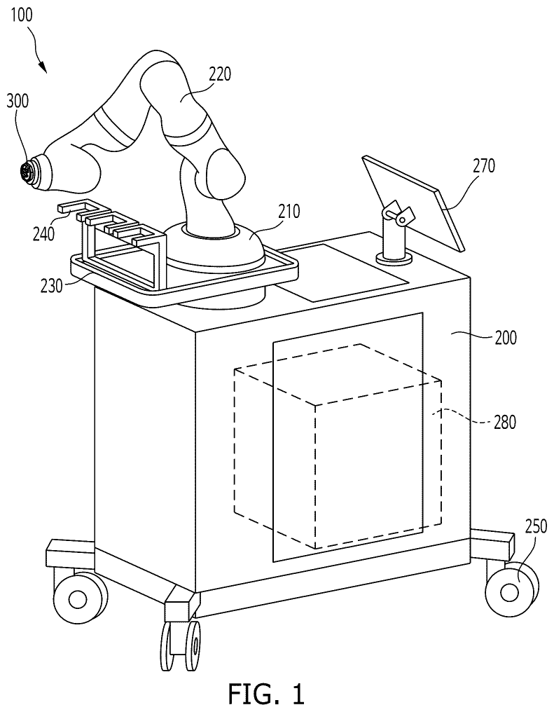

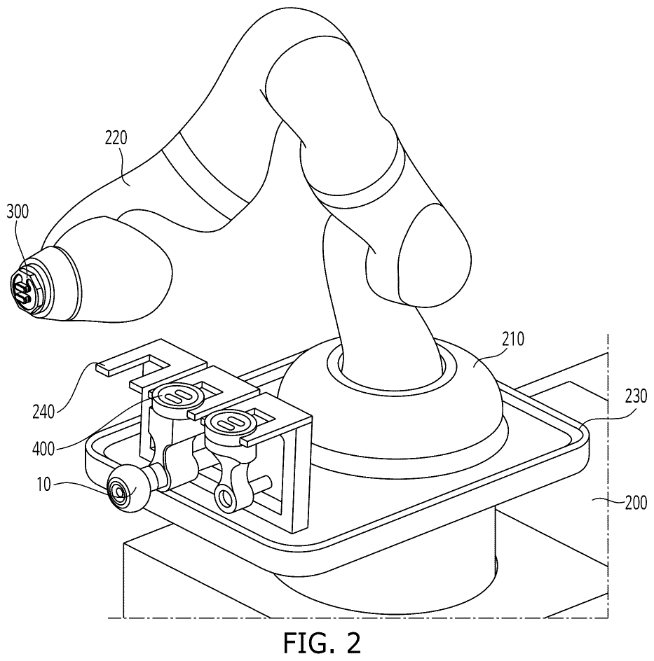

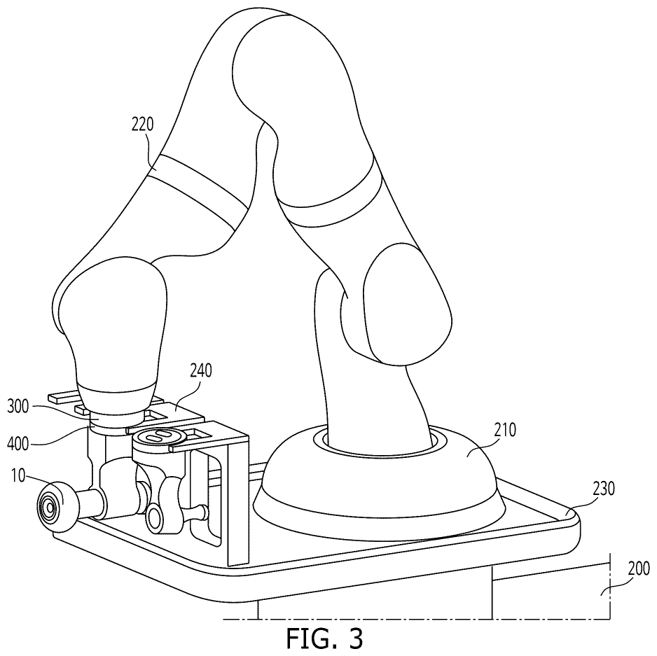

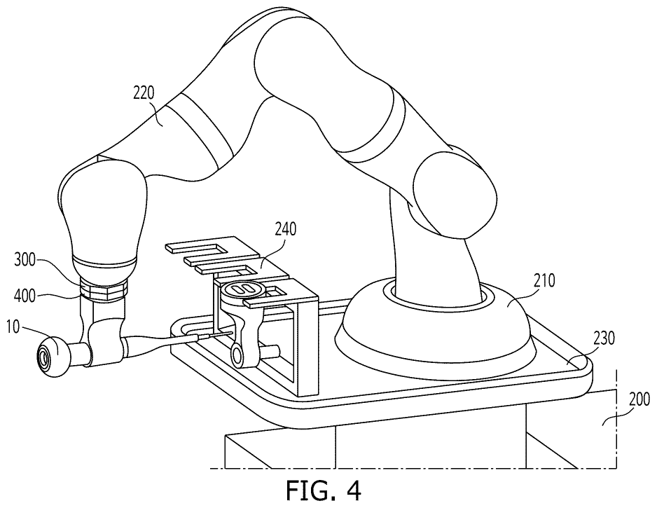

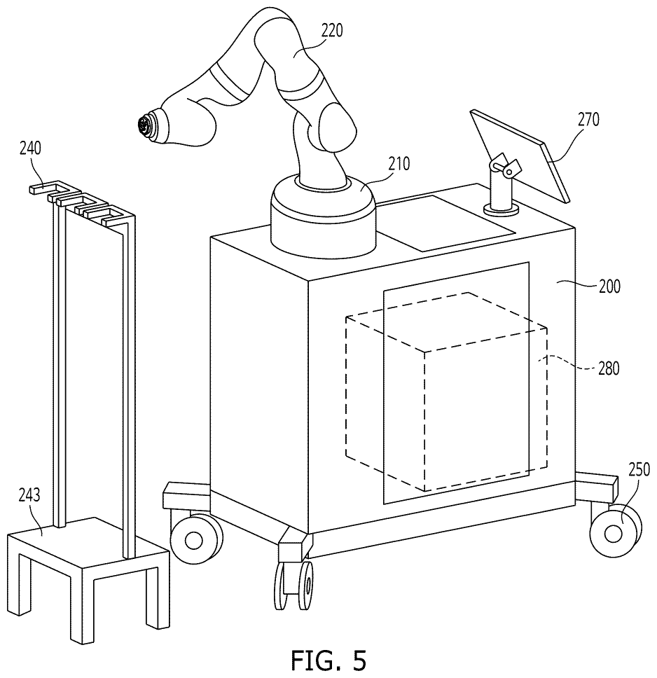

FIG. 1 is a perspective view illustrating an example of a surgical robot apparatus according to an embodiment of the present disclosure, FIG. 2 is a perspective view illustrating an enlarged view of the robot arm and the hanger, FIG. 3 is a perspective view illustrating an example in which the robot arm and the medical tool are coupled through a tool changer, FIG. 4 is a perspective view illustrating an example in which the medical tool coupled to the robot arm is separated from the hanger, and FIG. 5 is a perspective view illustrating another example of the hanger.

Referring to FIGS. 1 to 4, a surgical robot apparatus 100 according to an embodiment of the present disclosure may include a movable body portion 200, a robot arm 220 disposed on the body portion 200 to perform a predetermined operation, a first tool changer 300 coupled to the robot arm 220, a second tool changer 400 to which a medical tool 10 is coupled and which is attachable to and detachable from the first tool changer 300, a hanger 240 on which the second tool changer 400 is mounted, and a control unit 280 configured to control the operation of the robot arm 220.

The body portion 200 may be a structure in which the robot arm 220, the hanger 240, and the control unit 280 are disposed. In an embodiment, the body portion 200 may be formed in the shape of a box having a hollow. The control unit 280 may be disposed inside the body portion 200, and the robot arm 220 and the hanger 240 may be disposed on the upper part of the body portion 200.

The body portion 200 may be configured to be movable by a user. In an embodiment, the body portion 200 may further include a wheel portion 250 provided on the body portion 200 and configured to move the body portion 200. For example, when the body portion 200 is formed in the shape of a rectangular block, the wheel portion 250 may be provided at each of four corners of the body portion 200 so that a user may move the body portion 200 by pushing it. In another example, when the wheel portion 250 is configured to be driven by a motor, the control unit 280 may control the motor to move the body portion 200.

The robot arm 220 may be disposed on the body portion 200 and may perform a predetermined operation or perform command operations input to the control unit 280. The robot arm 220 may be operated by the control unit 280. An operation process of the robot arm 220 may be stored in advance in the control unit 280, and when a user inputs an execution command to the control unit 280, the control unit 280 may control the robot arm 220 to operate according to the stored operation process. Alternatively, when a user inputs an operation command for the robot arm 220 to the control unit 280, the control unit 280 may control the robot arm 220 to operate according to the input operation command.

The robot arm 220 may have a multi-joint structure. In an embodiment, the robot arm 220 may be configured as a robot arm having a 6-axis or 7-axis (6 or 7 Degrees of Freedom) joint structure, which allows high flexibility and precise manipulation.

The first tool changer 300 may be provided at an end of the robot arm 220 and may be attachable to and detachable from the second tool changer 400 to which the medical tool 10 is coupled. The first tool changer 300 may be coupled to the end of the robot arm 220 through a coupling member such as a screw.

The second tool changer 400 may be coupled to the medical tool 10 and may be attachable to and detachable from the first tool changer 300. The second tool changer 400 may be placed on the hanger 240 while being coupled to the medical tool 10.

In an embodiment, when the second tool changer 400 is placed on the hanger 240, the robot arm 220 may operate to preliminarily couple the first tool changer 300 to the second tool changer 400, and when the second tool changer 400 is released from the hanger 240, the first tool changer 300 and the second tool changer 400 may be coupled so that the robot arm 220 may be connected to the medical tool 10.

After the use of the medical tool 10 by the robot arm 220 is completed, the robot arm 220 may place the second tool changer 400 on the hanger 240. At this time, the coupling between the first tool changer 300 and the second tool changer 400 may be released, and the robot arm 220 may operate to separate the first tool changer 300 from the second tool changer 400. In addition, the second tool changer 400 may be fixed to the hanger 240.

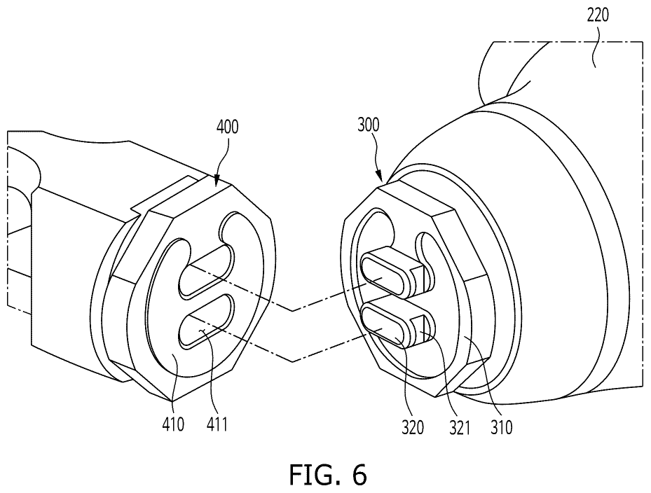

A detailed configuration of the first tool changer 300 and the second tool changer 400 will be described in detail with reference to FIGS. 6 to 8 below.

The surgical robot apparatus 100 may further include a display unit 270 disposed on the body portion 200 to input commands to the control unit 280 or to display an operation state of the robot arm 220. In an embodiment, the display unit 270 may be configured as a touch screen. A user may touch a command button displayed on the display unit 270 to input an operation command for the robot arm 220.

Of course, the configuration for inputting commands to the control unit 280 is not limited thereto, and the operation command of the robot arm 220 may be input using a keyboard and/or a mouse. The keyboard and mouse may be connected to the control unit 280 by wire or wirelessly.

The control unit 280 may control the operation of the robot arm 220. In an embodiment, the control unit 280 may be a computer device including an input/output interface connecting the robot arm 220 and the display unit 270 to the control unit 280 and mediating data input/output, a memory storing programs and data, a graphics processor performing image processing, a main processor performing arithmetic operations according to the programs and data stored in the memory, and a system bus serving as a path for data transmission and reception between components.

The control unit 280 may control the operation of the robot arm 220 so that the first tool changer 300 is coupled to the second tool changer 400 according to preset location data of the hanger 240. In an embodiment, location data of the hanger 240 may be stored in advance in the control unit 280. When use of the medical tool 10 is required, the control unit 280 may control the operation of the robot arm 220 to couple the first tool changer 300 to the second tool changer 400 based on the stored location data. Accordingly, the robot arm 220 may be connected to the medical tool 10 and may control the medical tool 10 through the tool changers 300 and 400.

When use of the medical tool 10 by the robot arm 220 is completed, the control unit 280 may mount the second tool changer 400 on the hanger 240 while the first tool changer 300 and the second tool changer 400 are coupled. In addition, the control unit 280 may control the operation of the robot arm 220 to separate the first tool changer 300 from the second tool changer 400.

The hanger 240 may be disposed on the body portion 200 and may be a structure on which the second tool changer 400 to which the medical tool 10 is coupled is mounted. Multiple hangers 240 may be disposed on the body portion 200. In an embodiment, referring to FIG. 2, three hangers 240 may be disposed on the body portion 200. A plurality of second tool changers 400 to which different medical tools 10 are coupled may be mounted on the respective three hangers 240. Of course, the number of hangers 240 is not limited thereto and may be two, four, or more.

The surgical robot apparatus may further include a support portion 210 provided on the body portion 200 and coupled to the robot arm 220, and a plate 230 coupled to the support portion 210 and on which the hanger 240 is disposed.

The support portion 210 may protrude to the outside of the body portion 200 and may be formed to surround a lower end of the robot arm 220. The support portion 210 may be a structure that supports the robot arm 220.

The plate 230 may have one side coupled to the support portion 210 and may be configured to have the hanger 240 disposed on the other side. The plate 230 may also have surgical tools used during surgery placed thereon.

In an embodiment, referring to FIG. 5, the hanger 240 may be disposed separately from the body portion 200. The hanger 240 may be configured to be movable independently of the body portion 200 and may include a support frame 243 to be placed on the ground. Therefore, the hanger 240 may be disposed at the front, rear, or side of the body portion 200.

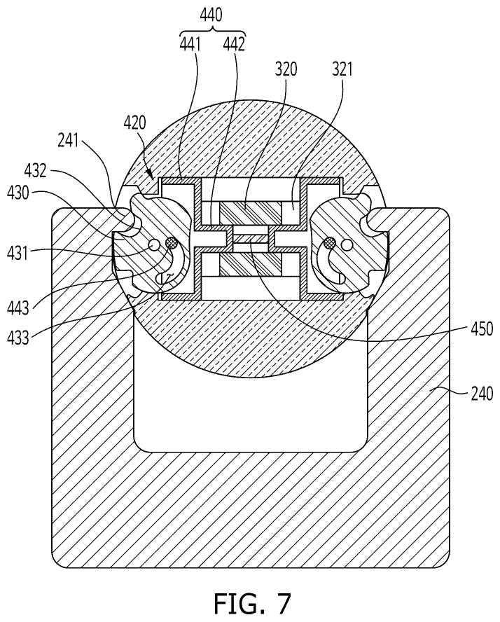

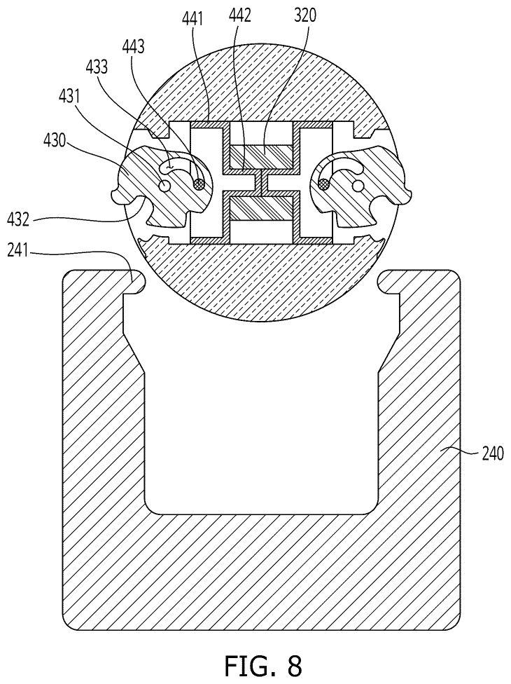

FIG. 6 is an enlarged view illustrating an example of the first tool changer coupled to the robot arm and the second tool changer coupled to the medical tool in the surgical robot apparatus according to an embodiment of the present disclosure, FIG. 7 is a cross-sectional view illustrating an example in which the first tool changer is placed on the second tool changer coupled to the hanger, and FIG. 8 is a cross-sectional view illustrating an example in which the second tool changer is separated from the hanger and is coupled to the first tool changer.

Referring to FIGS. 6 to 8, the first tool changer 300 may be provided at an end of the robot arm 220, and the second tool changer 400 may be connected to the medical tool 10. The first tool changer 300 and the second tool changer 400 may be coupled to be attachable and detachable.

The first tool changer 300 may include a first tool changer body 310 coupled to the robot arm 220 and a coupling protrusion 320 protruding from the first tool changer body 310 and inserted into and coupled to the second tool changer 400, the coupling protrusion 320 being formed with a coupling groove 321.

The first tool changer body 310 may be coupled to the end of the robot arm 220 through a coupling member such as a screw. The coupling protrusion 320 may protrude outward from the first tool changer body 310 and may be inserted into an insertion groove 411 formed in the second tool changer 400. In an embodiment, the coupling protrusion 320 may be formed in a columnar shape having an elliptical, circular, or rectangular cross-section. A plurality of coupling protrusions 320 may be provided on the first tool changer body 310. In an embodiment, two elliptical coupling protrusions 320 may be provided and may be arranged parallel to each other. A coupling groove 321 may be formed on both sides of the coupling protrusion 320 to be coupled to the second tool changer 400.

The second tool changer 400 may include a second tool changer body 410 to which the medical tool 10 is coupled and into which the coupling protrusion 320 is inserted, and a locking portion 420 provided on the second tool changer body 410 to be coupled to the hanger 240 or the first tool changer 300.

The second tool changer body 410 may be coupled to the medical tool 10 through a coupling member such as a screw. The locking portion 420 may be coupled to the hanger 240 when the second tool changer 400 is placed on the hanger 240. On the other hand, the locking portion 420 may be operated to be coupled to the first tool changer 300 when the second tool changer 400 is separated from the hanger 240.

In an embodiment, the locking portion 420 may include a locking lever 430 rotatably disposed on the second tool changer body 410 and restrained by an engagement protrusion 241 provided on the hanger 240 to rotate, a locking block 440 slidably disposed on the second tool changer body 410 and inserted into or separated from the coupling groove 321 in response to the rotation of the locking lever 430, and an elastic member 450 coupled to the locking block 440 applying an elastic force to insert the locking block 440 into the coupling groove 321.

The hanger 240 may include a locking protrusion 241 formed at an end where the second tool changer 400 enters and exits. In an embodiment, the hanger 240 may be formed in a U-shape with an opening formed on one side where the second tool changer 400 enters and exits. The locking protrusion 241 may protrude toward the second tool changer 400 inserted into the opening at one end of the hanger 240 where the opening is formed.

The locking lever 430 may be rotatably coupled to the second tool changer body 410 through a rotation hinge 431 and may include a locking groove 432 restrained by the locking protrusion 241 of the hanger 240. When the second tool changer 400 moves in the direction of insertion into the hanger 240, the locking protrusion 241 of the hanger 240 may be inserted into the locking groove 432 of the locking lever 430, thereby rotating the locking lever 430 in a first direction. When the second tool changer 400 moves in the direction of separation from the hanger 240, the locking protrusion 241 of the hanger 240 may be separated from the locking groove 432 of the locking lever 430, thereby rotating the locking lever 430 in a second direction opposite to the first direction.

The locking lever 430 may include an arc-shaped guide groove 433. The guide groove 433 may be configured such that a guide hinge 443 provided on the locking block 440 is inserted.

With this configuration, when the locking lever 430 rotates, the guide hinge 443 may move along the guide groove 433, causing the locking block 440 to move linearly. In an embodiment, when the locking lever 430 rotates in the first direction, the locking block 440 may move in a direction to be separated from the coupling groove 321. Also, when the locking lever 430 rotates in the second direction, the locking block 440 may move in a direction to be coupled to the coupling groove 321.

In an embodiment, the locking block 440 may include a locking block body 441 having the guide hinge 443 and moving linearly in association with the rotation of the locking lever 430 to be inserted into or separated from the coupling groove 321, and a locking block projection 442 protruding from the locking block body 441 and positioned between a pair of coupling protrusions 320. For example, the locking block 440 may be formed in an overall T-shape.

Referring to FIG. 7, in a state where the second tool changer 400 is inserted into the hanger 240, the locking block 440 may be separated from the coupling groove 321 of the coupling protrusion 320, so that the coupling protrusion 320 of the first tool changer 300 may be inserted into or separated from the insertion groove 411 of the second tool changer 400.

In a state where the second tool changer 400 is inserted into the hanger 240, the coupling protrusion 320 of the first tool changer 300 may be inserted into the insertion groove 411 of the second tool changer 400. In this configuration, as illustrated in FIG. 8, when the second tool changer 400 is separated from the hanger 240, the locking lever 430 may rotate due to the locking protrusion 241 of the hanger 240, thereby sliding the locking block 440 to insert the locking block 440 into the coupling groove 321 of the coupling protrusion 320 and fixing the first tool changer 300 and the second tool changer 400 together. When the locking block 440 is inserted into the coupling groove 321 of the coupling protrusion 320, the elastic member 450 may maintain the inserted state of the locking block 440 into the coupling groove 321 by its elastic force.

In an embodiment, the locking portion 420 may include a pair of locking levers 430 and a pair of locking blocks 440. The pair of locking levers 430 and the pair of locking blocks 440 may be respectively arranged on both sides of the coupling protrusion 320, with the coupling protrusion 320 therebetween. The coupling grooves 321 are formed on both sides of the coupling protrusion 320, so the pair of locking blocks 440 may be coupled to both sides of the coupling protrusion 320. At this time, the elastic member 450 may be coupled to connect the pair of locking blocks 440 to each other. The elastic member 450 may be coupled to apply an elastic force in a direction in which the pair of locking blocks 440 are inserted into the coupling grooves 321 of the coupling protrusion 320.

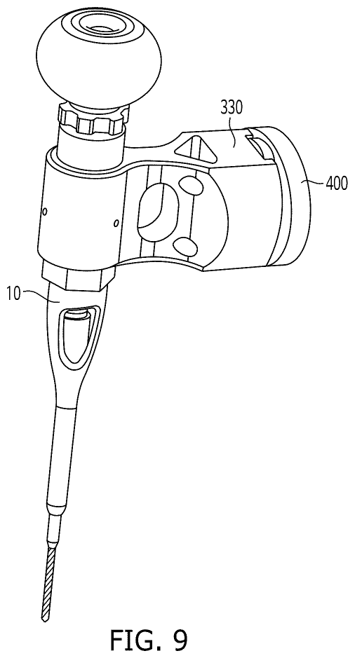

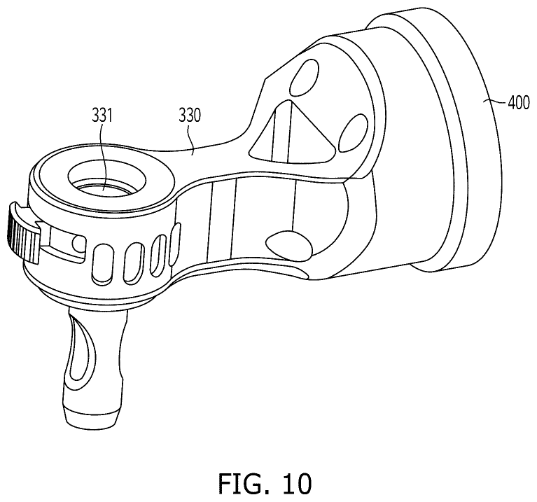

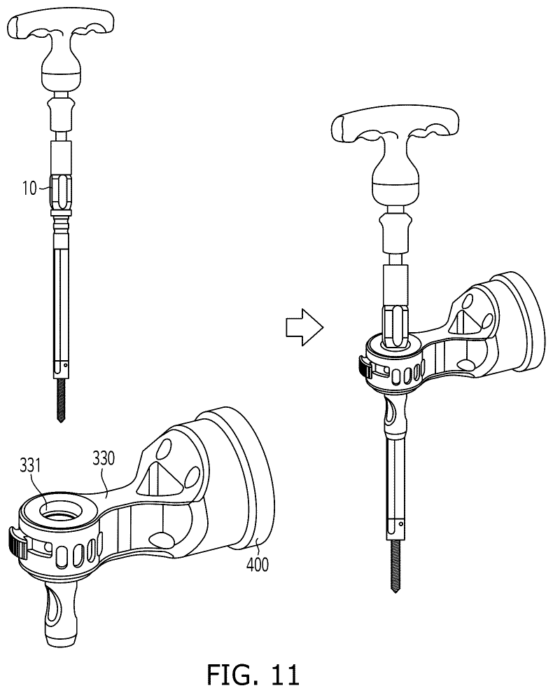

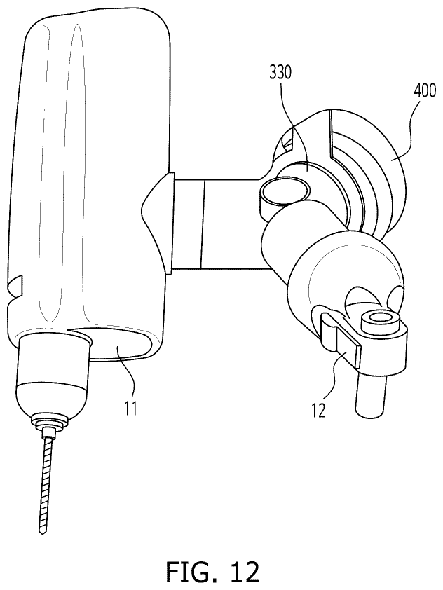

FIG. 9 is a perspective view illustrating an example of a coupling block integrally coupled with the medical tool in the surgical robot apparatus according to an embodiment of the present disclosure, FIG. 10 is a perspective view illustrating an example of the coupling block with the medical tool detachable, FIG. 11 is a perspective view illustrating an example in which the medical tool is coupled to the coupling block of FIG. 10, and FIG. 12 is a perspective view illustrating an example of the coupling block integrally coupled with two medical tools.

Referring to FIG. 9, the second tool changer 400 may be coupled to the medical tool 10 through a coupling block 330. In an embodiment, the second tool changer 400 may be coupled to one side of the coupling block 330, and the medical tool 10 may be integrally coupled to the other side of the coupling block 330 so as not to be separated. The second tool changer 400 may be coupled to the coupling block 330 through a coupling member such as a screw. Of course, the second tool changer 400 may also be integrally coupled to the coupling block 330 so as not to be separated.

As such, since the medical tool 10 is integrally coupled to the coupling block 330, it is possible to improve surgical precision by preventing a coupling error between the medical tool 10 and the coupling block 330. Here, the medical tool 10 may include an incision tool, a suture tool, a drill, a suction device, an endoscope, and the like.

In another embodiment, referring to FIGS. 10 and 11, the second tool changer 400 may be coupled to one side of the coupling block 330, and the medical tool 10 may be detachably coupled to the other side of the coupling block 330. That is, the medical tool 10 may be detachably coupled through a coupling portion 331 formed on the other side of the coupling block 330. The coupling portion 331 may include a ball joint coupling structure to prevent a coupling error with the medical tool 10. Of course, the form of the coupling portion 331 is not limited thereto, and various known coupling methods may be applied as long as the medical tool 10 can be coupled without error.

In another embodiment, referring to FIG. 12, the second tool changer 400 may be coupled to one side of the coupling block 330, and two medical tools 10 may be coupled to the other side of the coupling block 330. The first medical tool 11 and the second medical tool 12 coupled to the other side of the coupling block 330 may be tools performing different functions. In an embodiment, the first medical tool 11 and the second medical tool 12 may be tools that assist different functions in the same surgery. For example, the first medical tool 11 may be a drill, and the second medical tool 12 may be a guide for screw insertion. Thus, surgery may proceed more quickly using the two medical tools 10 coupled to one coupling block 330.

Although not shown in the drawings, various medical tools usable through the robot arm 220 may be coupled to the coupling block 330.

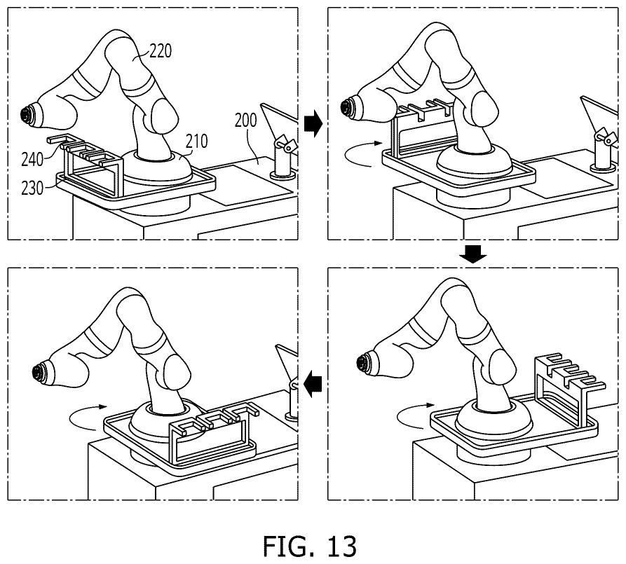

FIG. 13 is a perspective view illustrating an example in which the plate rotates to change the position of the hanger in the surgical robot apparatus according to an embodiment of the present disclosure.

Referring to FIG. 13, the plate 230 may have one side coupled to the support portion 210 and may be configured to have the hanger 240 disposed on the other side. The plate 230 may be rotatably coupled to the support portion 210. Therefore, the plate 230 may rotate around the support portion 210 to change the position of the hanger 240.

In an embodiment, the plate 230 may be configured to rotate around the support portion 210 by a preset angle such as 0 degrees, 90 degrees, 180 degrees, or 270 degrees. In another embodiment, the plate 230 may rotate 360 degrees around the support portion 210, and a user may rotate it to a desired arbitrary angle. The plate 230 may further include a locking member (not shown) to fix it so that it does not rotate further after being placed at a desired angle.

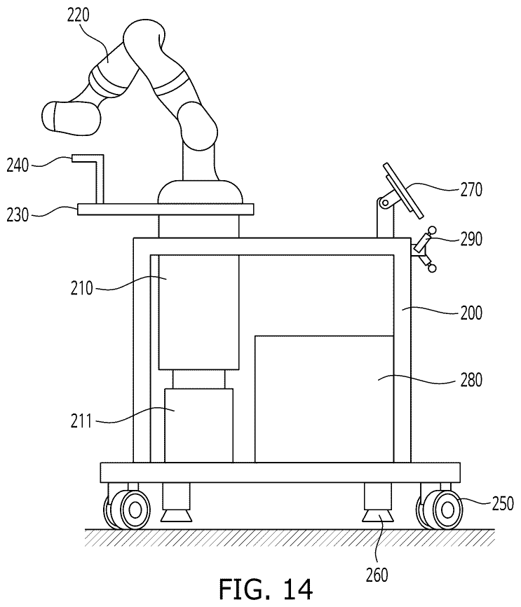

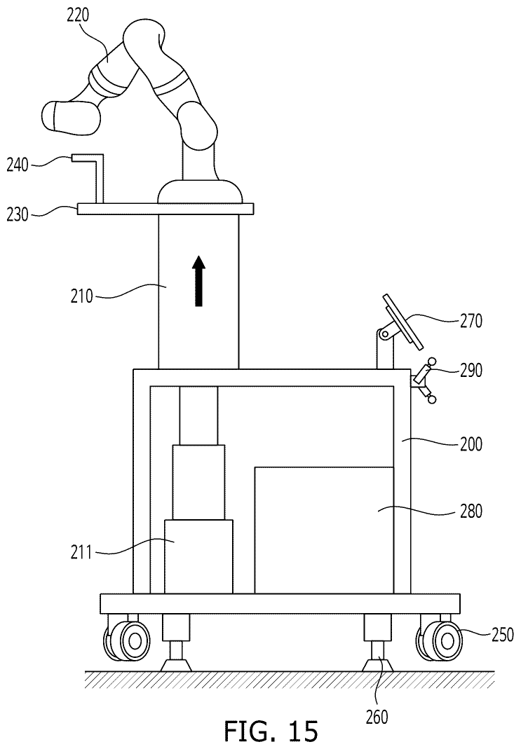

FIG. 14 is a side view illustrating an example of the surgical robot apparatus according to an embodiment of the present disclosure in which an actuator and a lifter are provided in the body portion, and FIG. 15 is a side view illustrating an example in which the actuator and the lifter are operated.

Referring to FIGS. 14 and 15, the surgical robot apparatus may further include an actuator 211 disposed in the body portion 200 to move the support portion 210 to adjust the height of the support portion 210, and a lifter 260 provided in the body portion 200 to selectively protrude and separate the wheel portion 250 from the ground to fix the position of the body portion 200.

The support portion 210 may be provided to slide through the body portion 200 and configured to protrude at least partially outside the body portion 200 to support the robot arm 220. In an embodiment, when the actuator 211 operates, the support portion 210 may protrude outward and be lifted upward from the body portion 200, and the robot arm 220 coupled to the support portion 210 may also move upward. Therefore, the height of the robot arm 220 may be adjusted by moving the support portion 210 vertically through the operation of the actuator 211.

The lifter 260 may be provided on the bottom surface of the body portion 200 and configured to selectively protrude to detach the wheel portion 250 from the ground, thereby fixing the position of the body portion 200. In an embodiment, a user may move the body portion 200 to a desired position by holding the handle 290 and then operate the lifter 260. Accordingly, the lifter 260 may protrude to lift the body portion 200 so that the wheel portion 250 is detached from the ground, thereby preventing the body portion 200 from moving by the wheel portion 250. That is, the lifter 260 may fix the position of the body portion 200 to prevent the body portion 200 from moving during a surgical procedure, thereby improving surgical precision.

In an embodiment, a user may input a command through the display portion 270, and the controller 280 may control the operation of the actuator 211 according to the received command to adjust the height of the robot arm 220. Additionally, the user may input a command through the display portion 270, and the controller 280 may control the operation of the lifter 260 according to the received command to fix the position of the body portion 200.

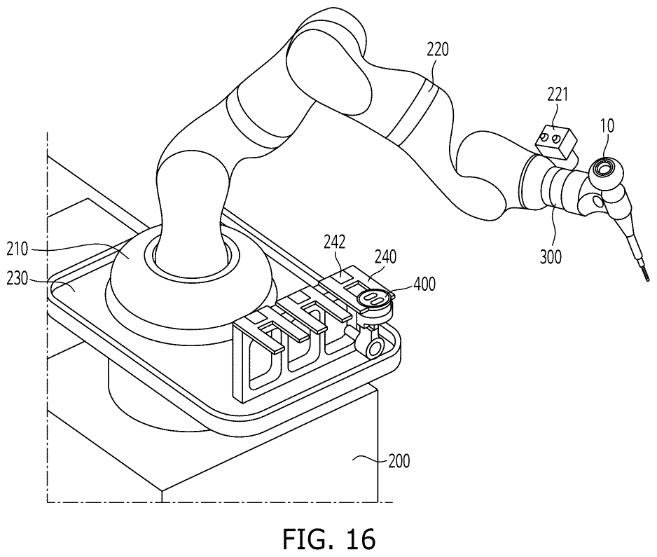

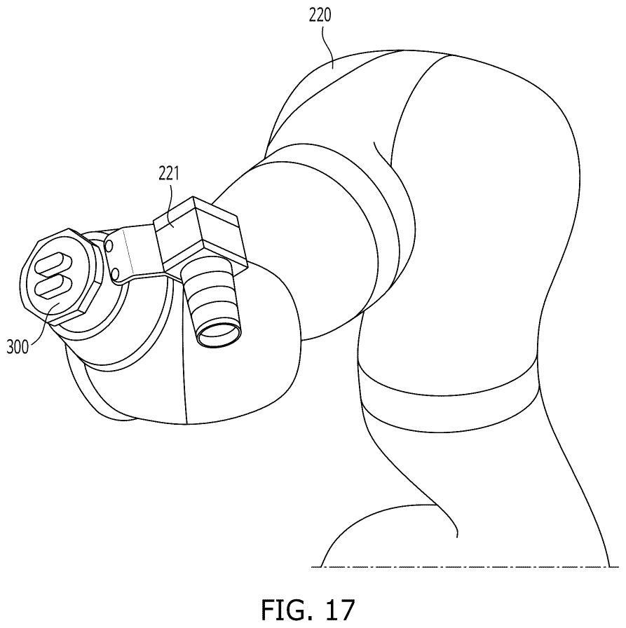

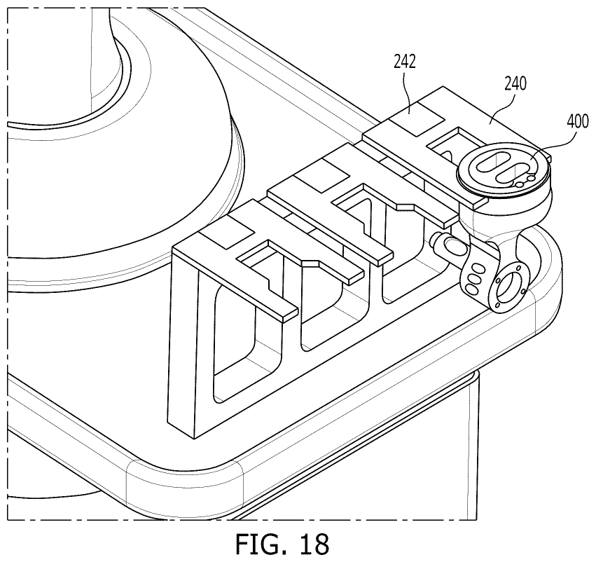

FIG. 16 is a perspective view illustrating an example of the surgical robot apparatus according to an embodiment of the present disclosure in which a vision sensor and a vision marker are provided, FIG. 17 is a perspective view illustrating an example in which the vision sensor is provided on the robot arm, and FIG. 18 is a perspective view illustrating an example in which the vision marker is provided on the hanger.

Referring to FIGS. 16 to 18, the surgical robot apparatus may further include a vision marker 242 provided on the hanger 240 and indicating the type of the medical tool 10 coupled to the second tool changer 400, and a vision sensor 221 provided on the robot arm 220 to recognize the vision marker 242.

The vision marker 242 may include a geometric pattern, a color combination, or a plurality of identifiable points. In an embodiment, the vision marker 242 may include an ArUco marker, a QR code, a barcode, and the like.

The vision sensor 221 may be coupled to the end of the robot arm 220 or to an area adjacent to the first tool changer 300. The viewpoint of the vision sensor 221 may move in association with the movement of the robot arm 220. In an embodiment, the vision sensor 221 may include a camera. The vision sensor 221 as a camera may photograph the vision marker 242 and transmit the captured image or video to the controller.

The controller may identify the position of the hanger 240 from the vision marker 242 photographed by the vision sensor 221 and control the operation of the robot arm 220 to couple the first tool changer 300 to the second tool changer 400.

In an embodiment, the controller may determine the relative position information of the hanger 240 from the position information of the vision marker 242 photographed by the vision sensor 221. The controller may calculate or identify the position information of the robot arm 220 in real-time based on the movement amount of the robot arm 220 and/or angle information of joints of the robot arm 220. For example, the controller may identify the initial position information of the robot arm 220. Here, the position information of the robot arm 220 may be the position and orientation of the first tool changer 300 coupled to the end of the robot arm 220. The controller may control the movement of the robot arm 220 while simultaneously updating the position information of the robot arm 220 in real-time.

The controller may receive an image of the vision marker 242 photographed by the vision sensor 221. The controller may calculate the angle and position information of the vision marker 242 from the received image and identify the position of the hanger 240 based on the calculated information. The hanger 240 does not move in the height direction, but a rotational position deviation may occur as the plate rotates. In this configuration, the position of the hanger 240 can be clearly identified from the image or video captured by the vision sensor 221.

The vision marker 242 provided on the hanger 240 may indicate the type of the medical tool 10 placed on the hanger 240. Therefore, the controller may identify the type of the medical tool 10 placed on the hanger 240 from the vision marker 242 photographed by the vision sensor 221 and may identify the medical tool 10 necessary for surgery. For example, different vision markers 242 may be provided on each of the plurality of hangers 240, and a medical tool corresponding to each vision marker 242 may be placed on each hanger 240.

In an embodiment, the marking of the vision marker 242 and the type of the corresponding medical tool may be stored in advance in the controller. A user may place a medical tool corresponding to the vision marker 242 attached to the hanger 240 on the hanger 240.

Of course, the method of identifying the type of the medical tool is not limited thereto, and the type of the medical tool may also be recognized by automatically analyzing the image of the medical tool captured by the vision sensor 221 using a deep learning-based image recognition model (e.g., YOLO).

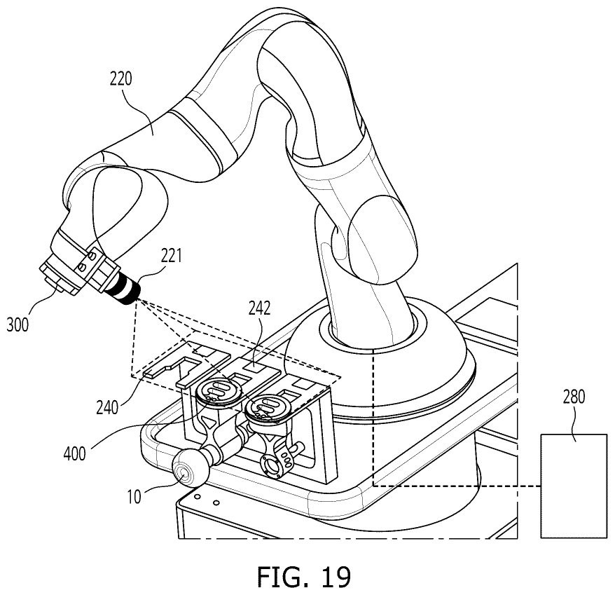

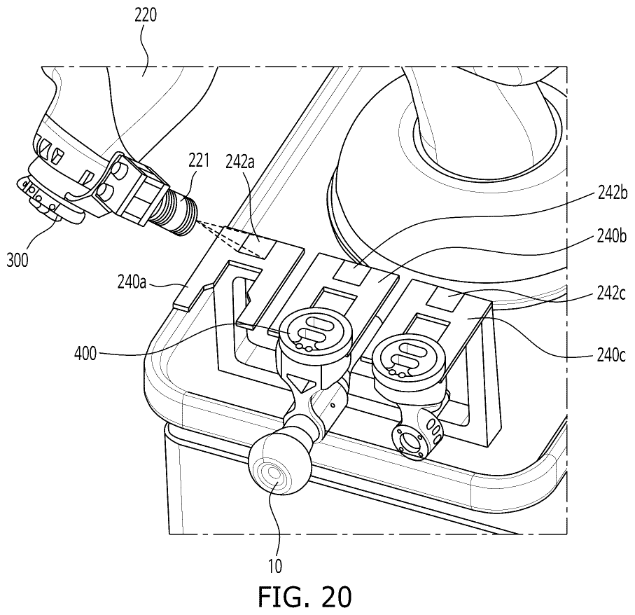

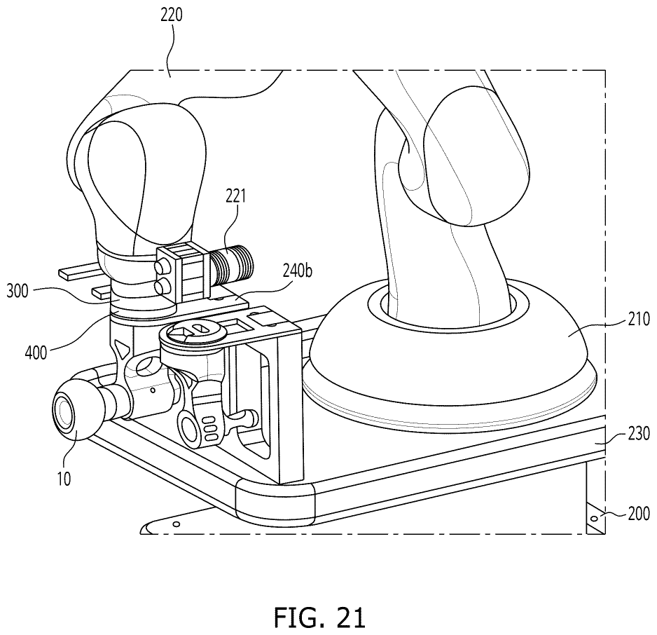

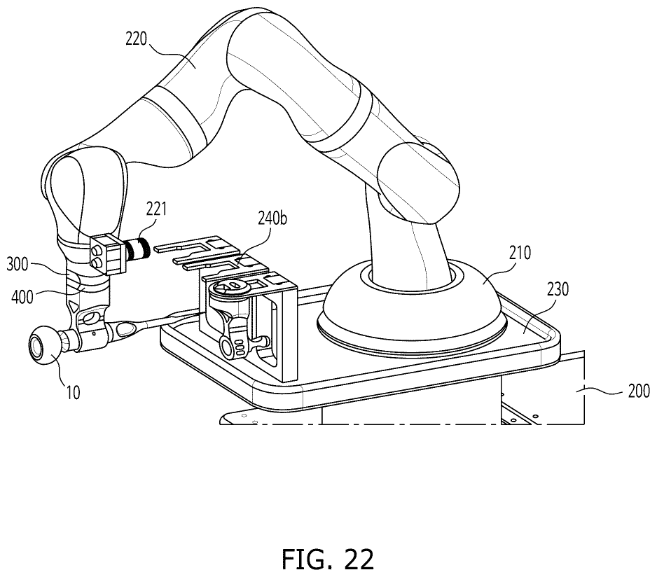

FIG. 19 is a perspective view illustrating an example of the surgical robot apparatus according to an embodiment of the present disclosure in which the vision sensor recognizes the vision marker to identify the position of the hanger, FIG. 20 is a perspective view illustrating an example in which the vision sensor recognizes the vision marker to identify the type of the medical tool provided on the hanger, FIG. 21 is a perspective view illustrating an example in which the first tool changer is coupled to the second tool changer after recognizing the vision marker, and FIG. 22 is a perspective view illustrating an example in which the medical tool coupled to the robot arm is separated from the hanger.

Referring to FIGS. 19 to 22, the hanger 240 may be provided in plurality, and a plurality of second tool changers 400 each coupled to different medical tools 10 may be coupled to the plurality of hangers 240. Additionally, the plurality of hangers 240 may each be provided with a plurality of vision markers 242.

The controller 280 may identify the positions and alignment directions of the plurality of hangers 240 from the plurality of vision markers 242 photographed by the vision sensor 221. That is, the controller 280 may calculate the angle and position information of the vision markers 242 from images of the plurality of vision markers 242 photographed by the vision sensor 221 and identify the positions of the plurality of hangers 240 based on the calculated information.

The controller 280, after identifying the positions of the plurality of hangers 240, may control the vision sensor 221 to photograph the vision markers 242 provided on the plurality of hangers 240, respectively. Then, the controller 280 may analyze the images photographed by the vision sensor 221 to identify the types of medical tools 10 placed on the plurality of hangers 240.

In an embodiment, the plurality of hangers may include a first hanger 240a, a second hanger 240b, and a third hanger 240c. The first hanger 240a may be provided with a first vision marker 242a, the second hanger 240b may be provided with a second vision marker 242b, and the third hanger 240c may be provided with a third vision marker 242c. Each of the first vision marker 242a to the third vision marker 242c may include a marker that can identify the type of the medical tool placed on each of the first hanger 240a to the third hanger 240c. For example, a user may place a medical tool corresponding to each of the first vision marker 242a to the third vision marker 242c on each of the first hanger 240a to the third hanger 240c in advance.

In this configuration, the controller 280 may identify the positions of the first hanger 240a to the third hanger 240c. Additionally, the controller 280 may recognize the first vision marker 242a to the third vision marker 242c provided on each of the first hanger 240a to the third hanger 240c and identify the types of medical tools provided on each of the first hanger 240a to the third hanger 240c.

Thereafter, the controller 280 may control the operation of the robot arm 220 to use the medical tool necessary for surgery. That is, the controller 280 may control the operation of the robot arm 220 to couple the first tool changer 300 to the second tool changer 400 coupled to the medical tool 10 required for the preset operation among the plurality of second tool changers 400.

For example, when using the medical tool 10 placed on the second hanger 240b, the controller 280 may operate the robot arm 220 to couple the first tool changer 300 of the robot arm 220 to the second tool changer 400 placed on the second hanger 240b, as illustrated in FIG. 21. Then, with the first tool changer 300 coupled to the second tool changer 400, the controller 280 may operate the robot arm 220 to separate the second tool changer 400 from the second hanger 240b, as illustrated in FIG. 22. Thereafter, the robot arm 220 may perform surgery using the medical tool 10 coupled to the first tool changer 300.

Furthermore, when the use of the medical tool 10 of the second tool changer 400 coupled to the first tool changer 300 is completed, the controller 280 may couple the second tool changer 400 to the hanger 240. In this state, the controller 280 may control the operation of the robot arm 220 to couple the first tool changer 300 to the second tool changer coupled to the medical tool required for the next preset operation. For example, when the use of the medical tool of the second tool changer 400 placed on the second hanger 240 is completed, the controller 280 may control the robot arm 220 to couple the second tool changer 400 to the second hanger 240. After the first tool changer 300 is separated from the second tool changer 400 coupled to the second hanger 240, the robot arm 220 may operate to couple the first tool changer 300 to the second tool changer that is placed on the third hanger 240 such that the medical tool placed on the third hanger 240 is used.

The vision marker may be provided on the hanger 240 and/or the second tool changer 400. The embodiment in which the vision marker is provided on the hanger 240 has been described in detail with reference to FIGS. 16 to 22.

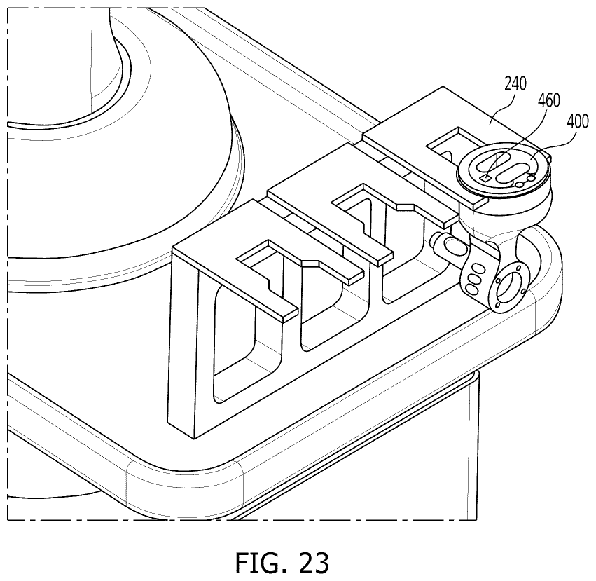

FIG. 23 is a perspective view illustrating an example of the surgical robot apparatus according to an embodiment of the present disclosure in which the vision marker is provided on the second tool changer.

Referring to FIG. 23, the vision marker 460 may be provided on the second tool changer 400. In an embodiment, the vision marker 460 may be provided on a surface of the second tool changer 400 where the first tool changer 300 is coupled.

The vision marker 460 provided on the second tool changer 400 may indicate the type of the medical tool coupled to the second tool changer 400. Therefore, the controller may identify the type of the medical tool coupled to the second tool changer 400 from the vision marker 460 photographed by the vision sensor and identify the medical tool required for surgery. For example, different medical tools may be placed on each of the plurality of hangers 240, and a vision marker 460 corresponding to the medical tool may be provided on each second tool changer 400 coupled to each medical tool.

Furthermore, the controller may calculate the angle and position information of the vision marker 460 from the image of the vision marker 460 photographed by the vision sensor and identify the position of the second tool changer 400 based on the calculated information. Therefore, the controller may identify the position of the second tool changer 400 and control the operation of the robot arm to couple the first tool changer 300 provided on the robot arm to the second tool changer 400.

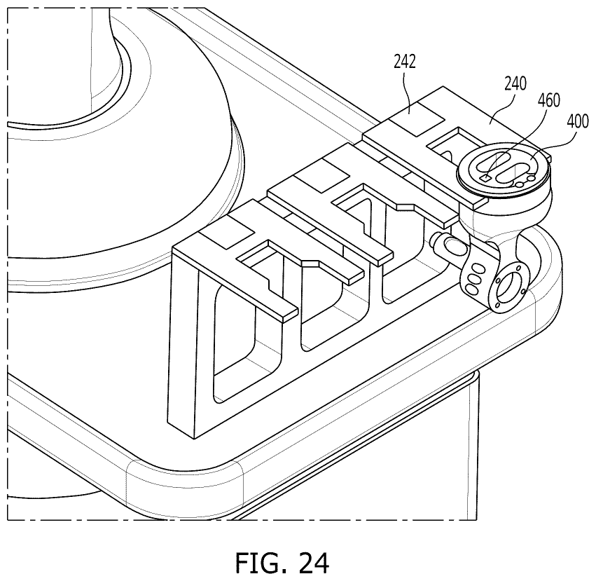

FIG. 24 is a perspective view illustrating an example of the surgical robot apparatus according to an embodiment of the present disclosure in which the vision marker is provided on both the hanger and the second tool changer.

Referring to FIG. 24, the hanger 240 may be provided with a position vision marker 242, and the second tool changer 400 may be provided with a tool vision marker 460.

In an embodiment, the position vision marker 242 provided on the hanger 240 may provide position information of the hanger 240. For example, the controller may calculate the angle and position information of the position vision marker 242 from the image of the position vision marker 242 photographed by the vision sensor and identify the position of the hanger 240 based on the calculated information.

In an embodiment, the tool vision marker 460 provided on the second tool changer 400 may provide information on the type of the medical tool coupled to the second tool changer 400. For example, the controller may identify the type of the medical tool placed on the hanger 240 from the image of the tool vision marker 460 photographed by the vision sensor.

With this configuration, the controller may first identify the position information of the hanger 240 by photographing the position vision marker 242 through the vision sensor. The controller may also secondarily identify the type of the medical tool placed on the hanger 240 by photographing the tool vision marker 460 through the vision sensor. Based on the information identified as described above, the controller may control the robot arm to couple the first tool changer to the second tool changer coupled to the medical tool required for the preset operation.

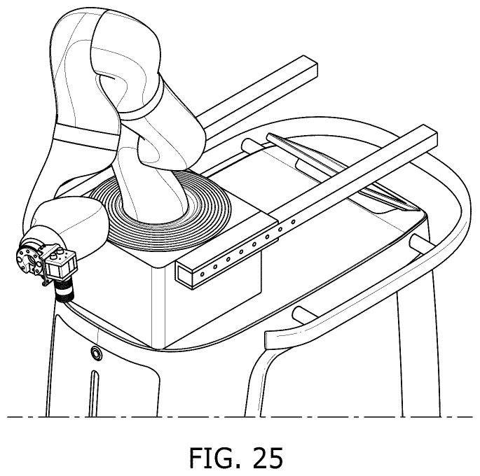

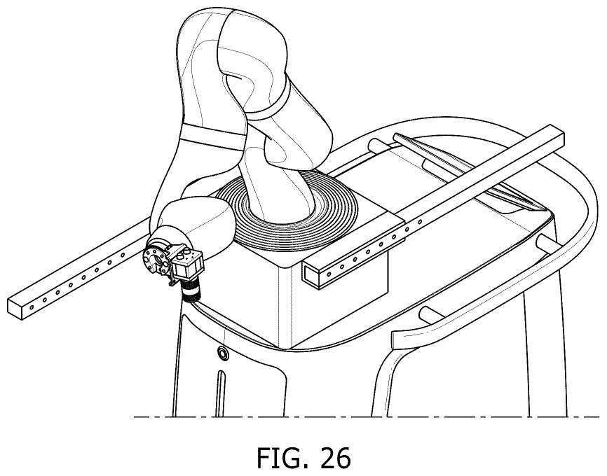

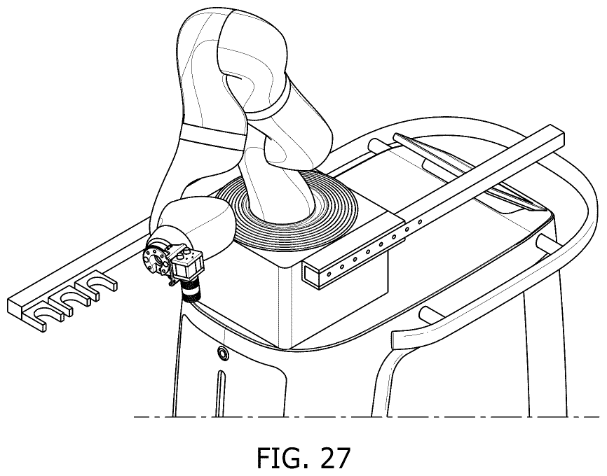

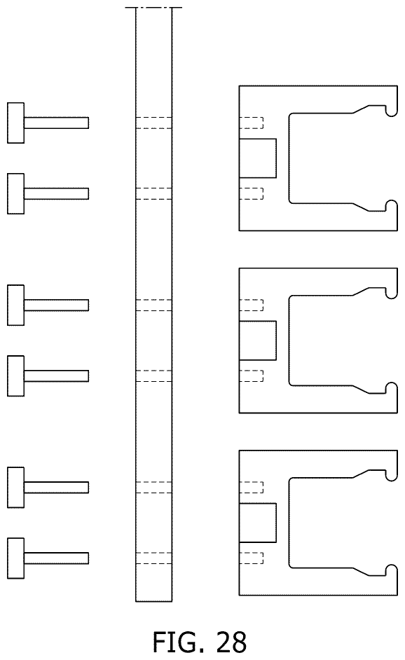

FIG. 25 is a perspective view illustrating an example of the surgical robot apparatus according to an embodiment of the present disclosure in which the hanger is detachably coupled to a slide bar, FIG. 26 is a perspective view illustrating an example in which the slide bar is slid, FIG. 27 is a perspective view illustrating an example in which the hanger is coupled to the slid slide bar, and FIG. 28 is a perspective view illustrating an example in which the hanger is coupled to the slide bar.

Referring to FIGS. 25 to 28, the surgical robot apparatus according to an embodiment of the present disclosure may include a support portion 510 provided in the body portion 200 and to which the robot arm 220 is coupled, and a slide bar 530 slidably coupled to the support portion 510.

The support portion 510 may protrude in the height direction from the upper surface of the body portion 200 and be formed to surround the lower end of the robot arm 220. The support portion 510 may be configured to support the robot arm 220. In an embodiment, the support portion 510 may be formed in a rectangular box shape.

The slide bar 530 may be coupled to the support portion 510 to slide in a direction parallel to the moving direction of the body portion 200. In an embodiment, the support portion 510 may be provided with a guide rail 520, and the slide bar 530 may be coupled to the guide rail 520 to slide. That is, the guide rail 520 and the slide bar 530 may be configured as a linear guide.

A plurality of slide bars 530 may be provided. In an embodiment, a plurality of slide bars 530 may be coupled to both sides of the support portion 510 and provided to slide in parallel directions.

The slide bar 530 may include a plurality of through holes 531 formed along its length. In an embodiment, referring to FIG. 26, the plurality of through holes 531 may be formed at predetermined intervals along the length of the slide bar 530 protruding outside the body portion 200.

With this configuration, as illustrated in FIG. 26, when the slide bar 530 is drawn out of the body portion 200, a hanger 550 may be coupled to the slide bar 530 using the through holes 531 formed in the slide bar 530.

In an embodiment, referring to FIG. 28, the hanger 550 may be coupled to the slide bar 530 through a coupling member 540 inserted into the plurality of through holes 531 of the slide bar 530. For example, the coupling member 540 may include a screw having threads formed on its outer circumferential surface. The hanger 550 may include a screw hole 551 to which the coupling member 540 is screw-coupled. After the coupling member 540 is inserted into the through hole 531 of the slide bar 530, the coupling member 540 may be screw-coupled to the screw hole 551 of the hanger 550 to couple the hanger 550 to the slide bar 530. Of course, the configuration for detachably coupling the hanger 550 to the slide bar 530 is not limited thereto, and various known fastening configurations may be applied.

With this configuration, when the hanger 550 is not used, the hanger 550 may be separated from the slide bar 530, and as illustrated in FIG. 25, the slide bar 530 may be arranged so as not to be drawn out of the outside of the body portion 200. That is, it is possible to prevent the slide bar 530 from interfering with the movement of the body portion 200 when the body portion 200 moves.

In addition, when the hanger 550 is used, as illustrated in FIG. 26, the slide bar 530 may be drawn out of the outside of the body portion 200, and as illustrated in FIG. 27, the hanger 550 may be coupled to the slide bar 530, and then a medical tool may be mounted on the hanger 550 and used.

The hanger 550 may be provided with a vision marker 242. In an embodiment, a vision sensor 221 provided on the robot arm 220 or at a third location (for example, an operating room) may photograph the vision marker 242 provided on the hanger 550 to identify the position of the hanger 550 or to identify the type of the medical tool mounted on the hanger 550. Of course, the present disclosure is not limited thereto, and the vision marker may also be provided on the second tool changer 400 or the slide bar 530 to provide position information of the hanger 550.

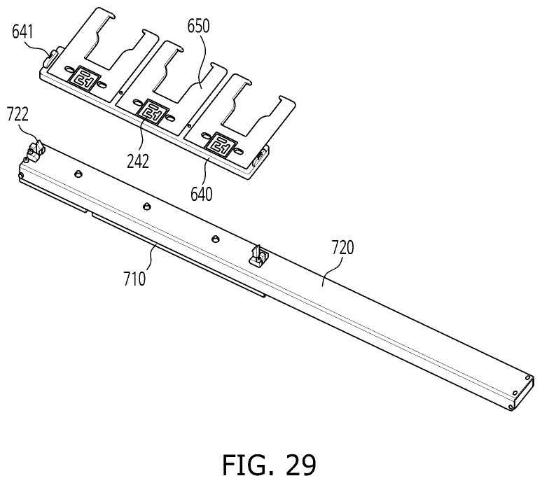

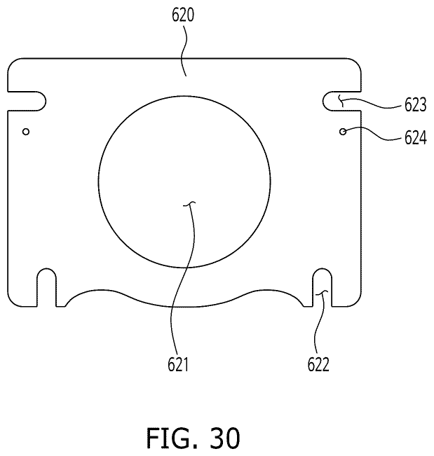

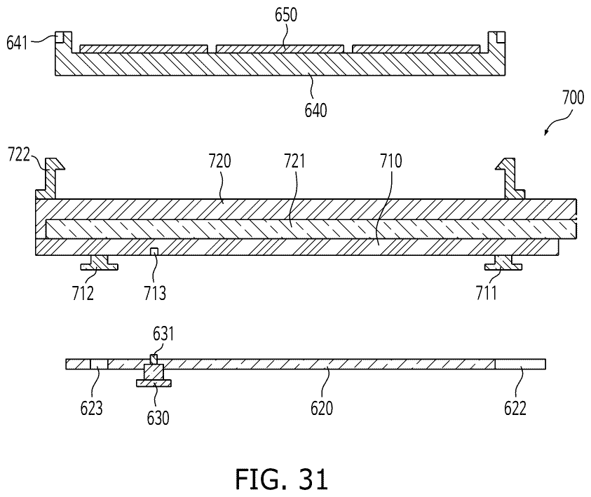

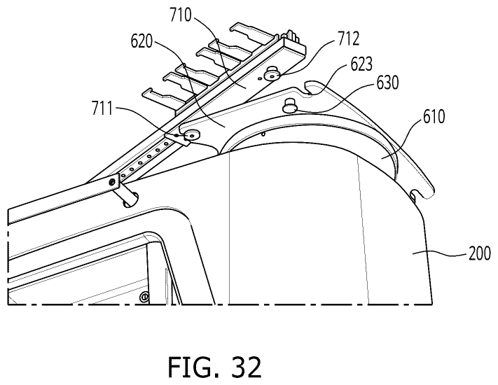

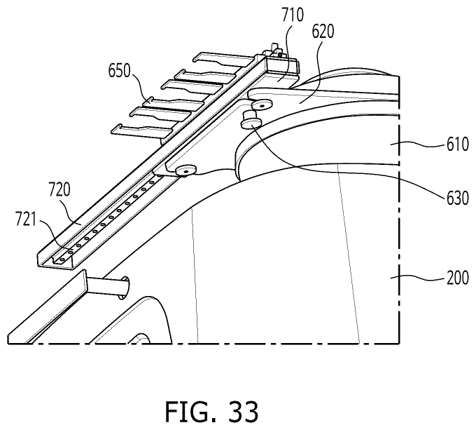

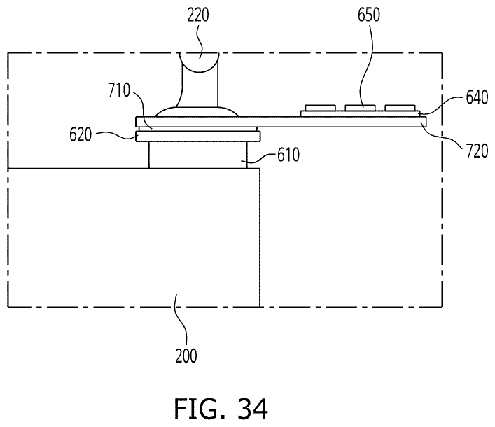

FIG. 29 is a perspective view illustrating an example of a slide portion to which a hanger is detachably coupled in a surgical robot apparatus according to an embodiment of the present disclosure, FIG. 30 is a plan view illustrating an example of a coupling plate to which the slide portion is detachably coupled, and FIG. 31 is an exploded cross-sectional view illustrating examples of the coupling plate, the slide portion, the hanger coupling portion, and the hanger. FIGS. 32 and 33 are perspective views illustrating examples in which the slide portion is coupled to the coupling plate, and FIG. 34 is a side view illustrating an example in which the slide bar is moved from the slide portion.

Referring to FIGS. 29 to 34, the surgical robot apparatus according to an embodiment of the present disclosure may include a support portion 610 provided on the body portion 200 and to which the robot arm 220 is coupled, a coupling plate 620 coupled to the support portion 610, at least one slide portion 700 detachably coupled to the coupling plate 620 and configured to slide, and a hanger coupling portion 640 detachably coupled to the slide portion 700 and provided with a hanger 650. The hanger coupling portion 640 may be provided with one or more hangers 650.

The support portion 610 may protrude in a height direction from an upper surface of the body portion 200 and may be formed to surround a lower end of the robot arm 220. The support portion 610 may be configured to support the robot arm 220. In an embodiment, the support portion 610 may be formed in a cylindrical shape.

The coupling plate 620 may be coupled to the support portion 610 and may be configured to support the slide portion 700. In an embodiment, the coupling plate 620 may be formed in an approximately rectangular plate shape, and a support portion coupling hole 621 may be formed through the center to couple to the support portion 610.

The slide portion 700 may be detachably coupled to the coupling plate 620, may be configured to slide, and may move the hanger coupling portion 640 to move the hanger 650 to a user-desired position. One slide portion 700 may be coupled to the coupling plate 620, or a pair of slide portions 700 may be coupled to both sides of the coupling plate 620.

In an embodiment, the slide portion 700 may include a slide body 710 including coupling protrusions 711 and 712 coupled to coupling holes 622 and 623 formed in the coupling plate 620, a slide bar 720 including a guide rail 721 coupled to the slide body 710 and configured for sliding movement, and coupling hooks 722 provided on the slide bar 720 and coupled to hook coupling grooves 641 formed at both ends of the hanger coupling portion 640.

The slide body 710 may be detachably coupled to the coupling plate 620 through the coupling protrusions 711 and 712 and may be configured to support the movement of the guide rail 721 moving together with the slide bar 720. In an embodiment, the slide body 710 may include a guide roller (not shown) guiding the sliding movement of the guide rail 721. That is, the slide body 710, the guide rail 721, and the slide bar 720 may be configured as a linear guide.

The coupling hooks 722 may be formed to protrude from the slide bar 720 and may be configured to couple the hanger coupling portion 640 to the slide bar 720. In an embodiment, a pair of hook coupling grooves 641 recessed inward at both ends of the hanger coupling portion 640 may be formed, and while the hanger coupling portion 640 is placed on an upper side of the slide bar 720, the pair of coupling hooks 722 formed on the slide bar 720 may be inserted into the pair of hook coupling grooves 641 and coupled.

In an embodiment, the coupling protrusions 711 and 712 formed on the slide body 710 may include a first coupling protrusion 711 formed on one side of the slide body 710 and a second coupling protrusion 712 formed on the other side of the slide body 710.

In an embodiment, the coupling holes 622 and 623 formed in the coupling plate 620 may include a first coupling hole 622 formed to penetrate outward from one side of the coupling plate 620 and a second coupling hole 623 formed to penetrate in a direction perpendicular to the first coupling hole 622 from the other side of the coupling plate 620.

With this configuration, as illustrated in FIG. 32, after the first coupling protrusion 711 is inserted into the first coupling hole 622, the slide body 710 may rotate toward the coupling plate 620, and the second coupling protrusion 712 may be inserted into the second coupling hole 623, thereby being coupled as illustrated in FIG. 33.

Here, the first coupling protrusion 711 and the second coupling protrusion 712 may have ends expanded outward in a radial direction so as to be caught and fastened in a state where they are inserted into the first coupling hole 622 and the second coupling hole 623, respectively. For example, the first coupling protrusion 711 and the second coupling protrusion 712 may have a T shape.

In a state where the coupling protrusions 711 and 712 of the slide body 710 are coupled to the coupling holes 622 and 623 of the coupling plate 620, a coupling member 630 may further be included to restrict the slide body 710 from rotating. That is, when the first coupling protrusion 711 is inserted into the first coupling hole 622 and the second coupling protrusion 712 is inserted into the second coupling hole 623 and the slide body 710 is restricted from rotating, the coupling state may be maintained so that the slide body 710 is not separated from the coupling plate 620.

The coupling member 630 may be coupled to the coupling plate 620 and may include a coupling pin 631 inserted into a pin coupling groove 713 formed in the slide body 710. In an embodiment, the coupling member 630 may be screw-coupled to the coupling plate 620, and the coupling pin 631 may be configured to be inserted into the pin coupling groove 713 while penetrating a coupling pin insertion hole 624 formed in the coupling plate 620.

In an embodiment, the coupling member 630 may be coupled to the coupling plate 620, and the coupling pin 631 may be elastically supported to the coupling member 630 by a spring (not shown). When an external force is applied to the coupling pin 631 as the slide body 710 rotates, the coupling pin 631 may be inserted into the coupling pin insertion hole 624, and when the pin coupling groove 713 is positioned on the coupling pin 631, the coupling pin 631 may be inserted into the pin coupling groove 713 by an elastic force of the spring.

With this configuration, after the first coupling protrusion 711 is inserted into the first coupling hole 622 and the second coupling protrusion 712 is inserted into the second coupling hole 623, the coupling pin 631 may be inserted into the pin coupling groove 713 to restrict the slide body 710 from rotating. When the coupling pin 631 is separated from the pin coupling groove 713 to release the rotation restriction of the slide body 710, the slide body 710 may be rotated to be separated from the coupling plate 620.

The hanger 650 may be provided with a vision marker 242. In an embodiment, a vision sensor provided on the robot arm 220 may photograph the vision marker 242 provided on the hanger 650 to identify the position of the hanger 650 or to identify the type of the medical tool mounted on the hanger 650. Of course, the present disclosure is not limited thereto, and the vision marker may also be provided on the slide bar 720 to provide position information of the hanger 650.

While the present disclosure has been described in connection with certain embodiments, various modifications and changes may be made without departing from the scope of the present disclosure as will be understood by those having ordinary skill in the art. Such modifications and changes should be considered as falling within the scope of the claims appended hereto.

What is claimed is:

1. A surgical robot apparatus comprising:
a movable body portion;
a robot arm disposed on the body portion to perform a predetermined operation;
a first tool changer coupled to the robot arm;
a second tool changer to which a medical tool is coupled and which is detachable from the first tool changer;
a hanger on which the second tool changer is mounted; and
a control unit for controlling an operation of the robot arm.

2. The surgical robot apparatus as claimed in claim 1, wherein the control unit controls the operation of the robot arm to couple the first tool changer to the second tool changer according to predetermined position data of the hanger.

3. The surgical robot apparatus as claimed in claim 2, wherein, in a state where the first tool changer and the second tool changer are coupled, the control unit controls the operation of the robot arm according to the predetermined position data of the hanger to mount the second tool changer on the hanger and then separate the first tool changer from the second tool changer.

4. The surgical robot apparatus as claimed in claim 1, wherein the first tool changer comprises:
a first tool changer body coupled to the robot arm; and
a coupling protrusion protruding from the first tool changer body, inserted into the second tool changer, and including a coupling groove coupled to the second tool changer.

5. The surgical robot apparatus as claimed in claim 4, wherein the second tool changer comprises:
a second tool changer body to which the medical tool is coupled and which includes an insertion hole into which the coupling protrusion is inserted; and
a locking part provided on the second tool changer body, coupled to the hanger when the hanger is disposed, and coupled to the first tool changer when detached from the hanger.

6. The surgical robot apparatus as claimed in claim 5, wherein the locking part comprises:
a locking lever rotatably disposed on the second tool changer body that is rotationally constrained by a locking protrusion provided on the hanger;
a locking block arranged to be slidingly movable on the second tool changer body and inserted into or separated from the coupling groove in response to the rotation of the locking lever; and
an elastic member engaged with the locking block and exerting elastic force to enable insertion of the locking block into the coupling groove.

7. The surgical robot apparatus as claimed in claim 1, further comprising a coupling block to which the second tool changer is coupled at one side and to which at least one medical tool is coupled at the other side.

8. The surgical robot apparatus as claimed in claim 1, further comprising:
a support portion provided on the body portion and to which the robot arm is coupled; and
at least one slide bar coupled to the support portion to slide in parallel to the direction in which the body portion moves,
wherein the hanger is detachably coupled to the slide bar.

9. The surgical robot apparatus as claimed in claim 8, wherein the slide bar includes a plurality of through-holes formed along a length direction, and
the hanger is detachably coupled to the slide bar by being coupled to or separated from a coupling member inserted into the plurality of through-holes.

10. The surgical robot apparatus as claimed in claim 1, further comprising:
a support portion provided on the body portion and to which the robot arm is coupled;
a coupling plate coupled to the support portion, at least one slide portion detachably coupled to the coupling plate and sliding; and
a hanger coupling portion detachably coupled to the slide portion and provided with the hanger.

11. The surgical robot apparatus as claimed in claim 10, wherein the slide portion comprises:
a slide body including a coupling protrusion coupled to a coupling hole formed in the coupling plate;
a slide bar including a guide rail coupled to the slide body and sliding; and
a coupling hook provided on the slide bar and coupled to a hook coupling groove formed at both ends of the hanger coupling portion.

12. The surgical robot apparatus as claimed in claim 11, wherein the coupling protrusion comprises a first coupling protrusion formed at one side of the slide body and a second coupling protrusion formed at the other side of the slide body,
the coupling hole comprises a first coupling hole formed to penetrate from one side of the coupling plate to the outside and a second coupling hole formed to penetrate from the other side of the coupling plate in a direction perpendicular to the first coupling hole, and
after the first coupling protrusion is inserted into the first coupling hole, the slide body rotates to insert the second coupling protrusion into the second coupling hole.

13. The surgical robot apparatus as claimed in claim 12, further comprising a coupling member coupled to the coupling plate and inserted into a pin coupling groove formed in the slide body,
wherein when the second coupling protrusion is inserted into the second coupling hole, the coupling pin is inserted into the pin coupling groove to restrain the slide body from rotating.

14. The surgical robot apparatus as claimed in claim 1, further comprising:
a vision marker provided on at least one of the hanger and the second tool changer and indicating the type of the medical tool coupled to the second tool changer, and
a vision sensor recognizing the vision marker.

15. The surgical robot apparatus as claimed in claim 14, wherein the control unit controls the operation of the robot arm to couple the first tool changer to the second tool changer by identifying the position of the hanger from the vision marker photographed by the vision sensor.

16. The surgical robot apparatus as claimed in claim 14, wherein the hanger is disposed in plurality,
a plurality of second tool changers each coupled with different medical tools are coupled to the plurality of hangers, and
a plurality of vision markers are provided on the plurality of hangers.

17. The surgical robot apparatus as claimed in claim 16, wherein the control unit identifies the positions and alignment directions of the plurality of hangers from the plurality of vision markers photographed by the vision sensor.

18. The surgical robot apparatus as claimed in claim 17, wherein the control unit identifies the types of the plurality of medical tools disposed on the plurality of hangers from the plurality of vision markers provided on the plurality of hangers.

19. The surgical robot apparatus as claimed in claim 18, wherein the control unit controls the operation of the robot arm to couple the first tool changer to the second tool changer coupled with the medical tool necessary for a predetermined operation among the plurality of second tool changers.

20. The surgical robot apparatus as claimed in claim 19, wherein the control unit controls the operation of the robot arm such that, when the use of the medical tool of the second tool changer coupled to the first tool changer is completed, the second tool changer is coupled to the hanger, and the first tool changer is coupled to a second tool changer to which the medical tool necessary for the set next operation is coupled.
